(19) United States
(12) Patent Application Publication
(10) Pub. No.: US 2012/0269908 A1
(43) Pub. Date: Oct. 25, 2012

(54) CARRIER IN OIL-IN-WATER EMULSION FORM, PARTICULARLY FOR COSMETIC OR DERMATOLOGICAL USE

(75) Inventors: Christian Sarrazin, Pegomas (FR); Marina Do, Menton (FR); Michele Boix, Cantaron (FR)

(73) Assignee: OCTALIA TECHNOLOGIES, Vallauris (FR)

(21) Appl. No.: 13/259,398

(22) PCT Filed: Nov. 27, 2009
(86) PCT No.: PCT/FR09/52323

§ 371 Date: Nov. 2, 2011

(30) Foreign Application Priority Data
Nov. 27, 2008 (FR) 0858067

(51) Int. Cl. A61K 9/107; A61K 47/32; A61K 47/14; A61K 47/30; A61K 47/36; A61K 47/38; A61K 38/17; A61K 31/17; A61K 31/70; A61K 36/00; A61K 31/573; A61P 17/00; A61P 31/00; A61P 31/10; A61P 33/00; A61P 17/10; A61P 17/04; A61P 17/12; A61P 17/06; A61K 8/06; A61K 8/64; A61K 8/60; A61K 8/40; A61K 8/97; A61K 47/34

(52) U.S. Cl. 424/725; 514/772; 514/772.2; 514/772.7; 514/785; 514/782; 514/777; 514/781; 514/772.4; 514/18.6; 514/588; 514/23; 514/169; 514/18.8

(57) ABSTRACT

The present invention relates to a carrier in oil-in-water emulsion form, containing a very small amount of a surface-active agent so that the emulsion is stable, well-tolerated, and nonirritating. The present invention also relates to a method for preparing a carrier according to the invention and to the use of such a carrier as a base for compositions to be applied onto sensitive human or animal body tissue, particularly in dermatological or dermocosmetic compositions.

This invention relates to a carrier in the form of a well-tolerated and non-irritating stable oil-in-water (O/W) emulsion, which contains a high oil phase proportion and a very small amount of surfactant.

This invention also relates to a method for preparing such a carrier and the use thereof as a base for cosmetic or dermatological compositions.

An emulsion is a mixture of two non-miscible liquids by mechanical agitation forming a dispersed system. This dispersion subsists as long as the agitation lasts, but once the agitation is stopped, globules coalesce and the liquids separate.

The formulation of an emulsion is intended to provide the mixture with the product(s) that will enable or facilitate the stabilization of the system. The product(s) are therefore essential for the formation and preservation in terms of physicochemical stability of an emulsion; they are called emulsifiers, surfactants, emulgators, wetting agents or surfactants and must be incorporated in a relatively large amount and generally indicated in dermatology and cosmetology as being capable of constituting up to 20% of the total weight of the emulsion in order to obtain adequate stability at a given temperature and over time.

However, surfactants are known for their irritating character, which significantly limits their use in dermo-cosmetic fields.

Thus, adverse effects associated with the use of surfactants are observed in the context of dermic applications, in particular allergenic effects, skin irritation (face, body, scalp), and these effects may be particularly irritating for dry and/or sensitive skin.

This problem is even worse when the emulsion is very high in oil, since it is necessary to similarly increase the proportion of surfactant so as to stabilize the composition and prevent phase changes thereof.

The technical problem to be solved therefore consists of producing emulsions containing a large proportion of oil but few surfactants, so that these emulsions have a satisfactory compromise between stability of the emulsion and tissue tolerance.

To overcome this technical problem, a person skilled in the art sought to eliminate the use of surfactant compounds or to reduce the amounts thereof by adding compounds intended to stabilize the emulsion such as viscosifying agents, fillers, and so on.

Thus, for example, the European patent application EP 1 095 659 describes a preparation in the form of an oil-in-water emulsion comprising oil, water and an alcohol constituting a hydro-alcohol phase, homeopathic and/or plant-based active principles and cellulose ether, which is known to a person skilled in the art for its emulsifying properties. Cellulose ether is present in the carrier of EP 1095659 in an amount of 0.1% to 10% by weight with respect to the total weight of the emulsion. In a particular example of the preparation of an emulsion of EP 1095659 containing a relatively high oil phase proportion (60% by weight with respect to the total weight of the emulsion) and consisting of medium-chain triglycerides, the proportion of cellulose ether is 1% by weight with respect to the total weight of the carrier. However, the emulsions of EP 1095659, which are emulsions stabilized by a cellulose either, must necessarily contain alcohol, in a proportion capable of ranging from 1 to 50% by weight of the final preparation. In the particular example cited above, the alcohol is present in an amount of at least 8% by weight with respect to the total weight of the carrier.

The preparations thus obtained indeed have good stability, but to the detriment of tissue compatibility insofar as the presence of alcohol in the preparation is known to cause irritation, very specifically in people with sensitive skin.

The European patent application EP 1 618 864 also describes oil-rich emulsions containing at least 60% oil phase by weight with respect to the total weight of the composition and containing an emulsifying system in an amount representing 2 to 20% by weight of the total weight of the composition. The mass ratio of the amount of emulsifying system on the amount of lipophilic phase varies from 0.04 to 0.2. To reinforce the physical stability of the system and contribute to a creamier texture, fillers are added in an amount of 0.5 to 10% by weight of the total weight of the preparation.

The international patent application WO 96/37180 describes pseudo-emulsion-type galenic compositions that can be used in dermatology and cosmetology, that do not use surfactants, but that involve thickening of the aqueous phase by the addition of gelling agents and thickening of the lipid phase by the use of glycerol esters. The latter are consistency factors that are semi-solid at room temperature and that confer, on the compositions obtained, microscopic structures different from those of emulsions. However, beyond a certain fatty phase amount (greater than 20%), the pseudo-emulsions thus obtained lose stability under heat and/or over time.

One objective of this invention is therefore to propose a carrier of the aforementioned type (O/W emulsion) comprising a high oil phase proportion, which overcomes the disadvantages of the prior art by incorporating only very small amounts of surfactant.

By high oil phase proportion in an O/W emulsion, we mean, in the sense of this invention, a proportion of at least 10% by weight of oil phase with respect to the total weight of the emulsion.

To achieve the objective of this invention, a carrier in oil-in-water (O/W) emulsion form comprising at least one aqueous phase (I) and at least one oil phase (II) is proposed, in which said aqueous phase (I) comprises at least one water-soluble surfactant and at least one water-soluble viscosifying agent.

According to the invention, the oil phase represents at least 10% by weight of the total weight of the carrier, and the water-soluble surfactant represents 0.1 to 0.5% by weight, and preferably 0.2 to 0.4% by weight of the total weight of the carrier.

The carrier according to the invention is stable, well tolerated and non-irritating.

Preferably, the oil phase is free of surfactant.

Indeed, the small amount of surfactant present in the aqueous phase is sufficient to ensure the stability of the emulsion and the carrier thus obtained is well tolerated and non-irritating. The addition of an additional surfactant in the oil phase does not provide anything in terms of improvement of the stability of the emulsion.

Advantageously, the oil phase represents no more than 60% by weight of the total weight of the carrier. Indeed, beyond this value, there is a risk of phase change of the emulsion that can occur several days after the emulsion has been produced, and this risk increases, for a given proportion of surfactants, when the oil phase proportion increases to beyond 60% by weight with respect to the total weight of the emulsion.

Preferably, the oil phase represents around 15% to 50% by weight of the total weight of the carrier. Indeed, for such a range of oil phase proportions, a good compromise is observed between a stable emulsion containing enough oil phase for a cosmetic or dermo-cosmetic application (in particular for dry skin) and good tissue tolerance.

With regard to the surfactant proportion, below 0.1% surfactant in the carrier according to the invention, it has the disadvantage of not containing enough surfactant to enable optimal ability to be achieved for an emulsion containing a high oil phase proportion.

Beyond 0.5% surfactant in the carrier according to the invention, it has the disadvantage of containing too much surfactant, and of compromising the tissue tolerance in particular for sensitive skin.

Preferably, the water-soluble surfactant is present in the carrier of the invention in an amount of 0.2% to 0.4% by weight with respect to the total weight of the carrier. In this preferred range (0.2 to 0.4% by weight of surfactant), a fine emulsion is obtained with oil globules having a mean size on the order of 2 μm to 5 μm.

As an example of a water-soluble surfactant that can be used in the carrier according to the invention, it is possible to cite in particular polysorbates, lecithins, polyethylene glycol derivatives, sorbitan esters, polyoxyethylene-polyoxypropylene copolymers and water-soluble polyvinyl alcohols.

Preferably according to the invention, a water-soluble polyvinyl alcohol (PVA) is used, and even more preferably a partially hydrolyzed polyvinyl alcohol, which is widely used in the pharmaceutical and cosmetic fields.

In addition, as a water-soluble viscosifying agent that can be used in the carrier according to the invention, it is possible to cite gums, glycosaminoglycans, cellulose and derivatives thereof, and acrylic polymers or carbomers.

It is possible to use, as water-soluble viscosifying agents, a xanthan sold by the CARGILL company under the trade name SATIAXANE®, an alginate sold by the CARGILL company under the trade name SATIALGINE®, a cross-linked polyacrylic acid sold by the Lubrizol company under the trade name CARBOPOL® 980NF.

The oil phase of the carrier according to the invention will now be described in greater detail.

The oil phase of the carrier according to the invention can contain one or more oils chosen from vegetable oils, mineral oils, silicone oils, synthetic oils and fluorinated oils.

Isopropyl myristate can in particular be cited as a synthetic oil capable of being used according to this invention.

Jojoba, avocado, sesame, sunflower, corn, soybean, safflower, grape seed, olive, almond, castor, moring a, coconut, palm, borage, rapeseed, wheat germ, linseed, primrose, argan calendula and cottonseed oils can be cited as vegetable oils capable of being used according to the invention.

Paraffin and preferably liquid paraffin oils can be cited as mineral oils capable of being used according to the invention.

A large fatty phase content is particularly beneficial for dermatological or cosmetic compositions intended for care of dry skin.

The carrier according to the invention can also include one or more compatible additives, which do not modify the characteristics specific to the emulsions.

Preservatives, antioxidants, isotonics, chelating agents, buffers, polymers, fillers, hydrophilic or lipophilic gelling agents, hydrophilic filters, hydrophilic compounds such as alcohols, odor absorbers, humectants and emollients, for example, can be cited as additives compatible with cosmetic and/or dermatological use.

Of course, a person skilled in the art will seek to choose any adjuvant(s) to be added to the compositions according to the invention, as well as the concentration thereof, so that the advantageous properties intrinsically associated with the compositions according to the invention are not substantially or at all altered by the addition envisaged.

In particular, these adjuvants must not adversely affect the properties of the composition according to the invention, i.e. good tolerance and lack of irritability of the skin.

This invention also relates to a cosmetic and/or dermo-cosmetic and dermatological composition including a carrier according to the invention, and at least one active principle for cosmetic and/or dermatological use.

The active principle for cosmetic and/or dermatological use can by water- and/or fat-soluble.

As active principles for dermatological use, it is possible to cite, for example, corticosteroids, antibiotics, antifungal agents, antiseptics, anti-parasitic agents, anti-herpetic agents, anti-acne agents, anti-pruritic agents, keratolitic agents, products for treating psoriasis, and products for treating atopic dermatitis.

As water-soluble active principles for cosmetic and/or dermo-cosmetic use capable of being used according to the invention, it is possible to cite in particular proteins, amino acids, polyols, urea, sugars and sugar derivatives, water-soluble vitamins, plant-based extracts and hydroxy acids.

As fat-soluble active principles for cosmetic and/or dermo-cosmetic use capable of being used according to the invention, it is possible to cite in particular UVA and UVB filters, and fat-soluble vitamins such as retinol (vitamin A) and its derivatives and tocopherol (vitamin E) and its derivatives.

This invention also relates to a method for preparing a carrier, including the steps consisting of:

a) preparing an oil-in-water emulsion by introducing, in a reactor, at least one oil that will represent 10% to 60% by weight of the total weight of the final carrier, then adding an aqueous surfactant solution in an amount so that:
the mass ratio of the amount of aqueous phase in the first emulsion over the amount of oil phase in the first emulsion is between 1/3 and 1/1, and the mass ratio of the amount of surfactant in the carrier (i.e. the final carrier obtained from step c) over the amount of oil in the carrier is between 0.007 and 0.020;

b) preparing an aqueous water-soluble viscosifying agent solution, of which the concentration is dependent on the viscosifying agent used and the intended use;

c) introduction of the first emulsion into the aqueous water-soluble viscosifying agent solution, in order to obtain a second emulsion constituting said carrier, in which the amount of first emulsion introduced into the aqueous water-soluble viscosifying agent solution is such that the oil phase in said carrier represents at least 10% by weight of the total weight of said carrier and the water-soluble surfactant represents 0.1% to 0.5% by weight of the total weight of said carrier.

According to one advantageous embodiment of the method of the invention, step a) of the method of the invention is performed so that the mass ratio of the amount of aqueous phase in the first emulsion over the amount of oil phase in the first emulsion is on the order of 1/2. In such an embodiment, the first emulsion obtained from step a) is thick enough but not too thick, so that it can easily be manipulated during production of the carrier.

The surfactant and the water-soluble viscosifying agent are as described above.

The invention will now be illustrated by means of the following non-limiting examples. The amounts are indicated as a percentage by weight unless otherwise indicated.

### EXAMPLES

### Products

### Oil Phase Components

vegetable oils: almond oil, sunflower oil, fat-soluble surfactant: polyglyceryl-3 dioleate (sold under the trade name Plurol Oleique CC497 by the Gattefosse company)

### Aqueous Phase Components

purified water
water-soluble surfactant: partially hydrolyzed polyvinyl alcohol (PVA) sold by the SIGMA company under the trade name MOWIOL® 40-88, water-soluble viscosifying agents: xanthan sold by the CARGILL company under the trade name SATIAXANE®; alginate sold by the CARGILL company under the trade name SATIALGINE®; cross-linked polyacrylic acid sold by the Lubrizol company under the trade name CARBOPOL® 980NF.

### Example 1

A first example of a carrier E1 according to the invention was prepared, containing 25% oil by weight with respect to the total weight of the carrier and 0.375% surfactant by weight with respect to the total weight of the carrier. Example 1 is prepared as follows.

A first emulsion (O/W) is first prepared by dispersion of 30 g of oil in 15 g of aqueous PVA solution at 3% by weight. The introduction of oil into the aqueous PVA solution is performed under high agitation (Ultra-turrax for 1.5 minute).

In addition, 62.5 g of aqueous solution containing 1.6% by weight of xanthan is prepared.

Then, 37.5 g of the first PVA-based emulsion (O/W) is added to the aqueous xanthan solution at 1.6% by weight, under agitation by means of a rotor, in order to obtain the final emulsion constituting a first example E1 of a carrier according to the invention. The final weight is adjusted with water.

The composition of the carrier E1 according to the invention is detailed in Table 1 below.

### Example 2

A second example E2 of a carrier according to the invention was prepared in the same was as in example 1, containing 18.8% of oil by weight of the total weight of the carrier and 0.14% of surfactant by weight of the total weight of the carrier. Example 2 is prepared as follows.

A first emulsion (O/W) is first prepared by dispersion of 22.5 g of oil in 22.5 g of aqueous PVA solution at 0.75% by weight. The introduction of oil into the aqueous PVA solution is performed under high agitation (Ultra-turrax for 1.5 minute).

In addition, 62.5 g of aqueous solution containing 1.6% by weight of xanthan is prepared.

Then, 37.5 g of the first PVA-based emulsion (O/W) is added to the aqueous xanthan solution at 1.6% by weight, under agitation by means of a rotor, in order to obtain the final emulsion constituting a second example E2 of a carrier according to the invention. The final weight is adjusted with water.

The composition of the carrier E2 according to the invention is detailed in Table 1 below.

### Example 3

A third example E3 of a carrier according to the invention was prepared in the same was as in example 1, containing 40% of oil by weight of the total weight of the carrier and 0.375% of surfactant by weight of the total weight of the carrier. Example 3 is prepared as follows.

A first emulsion (O/W) is first prepared by dispersion of 46.6 g of oil in 22.4 g of aqueous PVA solution at 1.9% by weight. The introduction of oil into the aqueous PVA solution is performed under high agitation (Ultra-turrax for 1.5 minute).

In addition, 40 g of aqueous solution containing 2.5% by weight of xanthan is prepared.

Then, 60 g of the first PVA-based emulsion (O/W) is added to the aqueous xanthan solution at 2.5% by weight, under agitation by means of a rotor, in order to obtain the final emulsion constituting a third example E3 of a carrier according to the invention. The final weight is adjusted with water.

The composition of the carrier E3 according to the invention is detailed in Table 1 below.

### Example 4

A fourth example E4 of a carrier according to the invention was prepared in the same was as in example 1, containing 18.8% of oil by weight of the total weight of the carrier and 0.375% of surfactant by weight of the total weight of the carrier, and by replacing the xanthan solution with an alginate solution. Example 4 is prepared as follows.

A first emulsion (O/W) is first prepared by dispersion of 22.5 g of oil in 22.5 g of aqueous PVA solution at 2% by weight. The introduction of oil into the aqueous PVA solution is performed under high agitation (Ultra-turrax for 1.5 minute).

In addition, 62.5 g of aqueous solution containing 3.2% by weight of xanthan is prepared.

Then, 37.5 g of the first PVA-based emulsion (O/W) is added to the aqueous xanthan solution at 3.2% by weight, under agitation by means of a rotor, in order to obtain the final emulsion constituting a fourth example E4 of a carrier according to the invention. The final weight is adjusted with water.

The composition of the carrier E4 according to the invention is detailed in Table 1 below.

### Example 5

A fifth example E5 of a carrier according to the invention was prepared in the same was as in example 1, containing 25% of oil by weight of the total weight of the carrier and 0.375% of surfactant by weight of the total weight of the carrier. Example 5 is prepared as follows.

A first emulsion (O/W) is first prepared by dispersion of 30 g of oil in 15 g of aqueous PVA solution at 3% by weight. The introduction of oil into the aqueous PVA solution is performed under high agitation (Ultra-turrax for 1.5 minute).

In addition, 62.5 g of aqueous solution containing 0.48% by weight of cross-linked polyacrylic acid adjusted with soda to between pH 6 and 7. Then, 37.5 g of the first PVA-based emulsion (O/W) is added to the aqueous cross-linked polyacrylic acid solution at 0.48% by weight, under agitation by means of a rotor, in order to obtain the final emulsion constituting a fifth example E5 of a carrier according to the invention. The final weight is adjusted with water.

The composition of the carrier E5 according to the invention is detailed in Table 1 below.

### Comparative Example EC1

A first example of a comparative composition containing 50% oil by weight of the total weight of the carrier and 0.375% surfactant by weight of the total weight of the carrier.

The comparative example is prepared as follows.

First, 58.3 g of oil is mixed in 11.7 g of aqueous PVA solution at 3.75% by weight. The introduction of oil in the aqueous PVA solution is performed under high agitation (Ultra-turrax for 1.5 minute). An intermediate composition that is not an emulsion is obtained. Indeed, in this intermediate composition, the mass ratio of the amount of aqueous phase in the first emulsion over the amount of oil phase is 1/5. With such a mass ratio, an immediate phase change of the composition, which has not been emulsified, is observed.

Then, 40 g of aqueous solution containing 2.5% by weight of xanthan is prepared.

Then, 60 g of the intermediate PVA-based composition is added to the aqueous xanthan solution at 2.5% by weight, under agitation by means of a rotor, in order to obtain a first comparative example EC1 of the carrier, of which the final weight is adjusted with water. Like the intermediate composition, the carrier EC1 is not an emulsion.

The composition of the carrier EC1 is presented in Table 1 below.

### Comparative Example EC2

A second example of a comparative composition containing 25% oil by weight of the total weight of the carrier and 0.05% surfactant by weight of the total weight of the carrier, and in which the mass ratio of the amount of surfactant in the final carrier over the amount of oil in the final carrier is no longer complied with. Comparative example 2 is prepared as follows.

A first emulsion (O/W) is first prepared by dispersion of 30 g of oil in 15 g of aqueous PVA solution at 0.4% by weight. The introduction of oil into the aqueous PVA solution is performed under high agitation (Ultra-turrax for 1.5 minute).

In addition, 62.5 g of aqueous solution containing 1.6% by weight of xanthan is prepared.

Then, 37.5 g of the first PVA-based emulsion (O/W) is added to the aqueous xanthan solution at 1.6% by weight, under agitation by means of a rotor, in order to obtain a second comparative example EC2 of the carrier according to the invention, of which the final weight is adjusted with water. The carrier EC2 is an unstable emulsion.

The composition of the carrier EC2 is presented in Table 1 below.

### Comparative Example EC3

A third example of a comparative composition containing 25% oil by weight of the total weight of the carrier and 0.376% surfactant by weight of the total weight of the carrier and 0.5% of fat-soluble surfactant by weight of the total weight of the carrier. Comparative example 3 is prepared as follows.

An oil phase consisting of 30 g of oil and 0.61 g of Plurol Oleique CC497 is first prepared. The, a first emulsion is produced by dispersion of 30.61 g of oil phase in 15 g of aqueous PVA solution at 3% by weight. The addition of the oil phase to the aqueous PVA solution is performed under high agitation (Ultra-turrax for 1.5 minute).

In addition, 62.0 g of aqueous solution containing 1.6% by weight of xanthan is prepared.

Then, 38.0 g of the first PVA-based emulsion (O/W) is added to the aqueous xanthan solution at 1.6% by weight, under agitation by means of a rotor, in order to obtain a third comparative example EC3 of the carrier according to the invention, of which the final weight is adjusted with water. The carrier EC3 is an unstable emulsion.

The composition of the carrier EC3 is presented in Table 1 below.

TABLE 1

| Composition | E1 (in g/ | E2 (in g/ | E3 (in g/ | E4 (in g/ | E5 (in g/ | EC1 (in | EC2 (in | EC3 (in |
|---|---|---|---|---|---|---|---|---|
| (in g/ for | for 100 g | for 100 g | for 100 g | for 100 g | for 100 g | g/ for | g/ for | g/ for |
| 100 g of | of | of | of | of | of | 100 g of | 100 g of | 100 g of |
| carrier) | carrier) | carrier) | carrier) | carrier) | carrier) | carrier) | carrier) | carrier) |
| PVA | 0.375 | 0.14 | 0.375 | 0.375 | 0.375 | 0.375 | 0.05 | 0.375 |
| xanthan | 1 | 1 | 1 | — | — | 1 | 1 | 1 |
| alginate | — | — | — | 2 | — | — | — | — |
| cross- | — | — | — | — | 0.3 | — | — | — |
| linked | | | | | | | | |
| polyacrylic | | | | | | | | |
| acid adjusted | | | | | | | | |
| to pH 6-7 | | | | | | | | |
| oil | 25 | 18.8 | 40 | 18.8 | 25 | 50 | 25 | 25 |
| polyglyceryl- | — | — | — | — | — | — | — | 0.5 |
| 3-dioleate | | | | | | | | |
| purified | qsf 100 g | qsf 100 g | qsf 100 g | qsf 100 g | qsf 100 g | qsf 100 g | qsf 100 g | qsf 100 g |
| water | | | | | | | | |

For all of the compositions according to the invention E1, E2, E3, E4, E5, EC1, EC2 and EC3, the characteristics of the following emulsion were evaluated:
macroscopic aspect,
measurement of the size of the oil globules in the carrier by laser granulometry; the size expressed in μm is reported for each sample.

The performance of the emulsion in centrifugation was also evaluated as a provisional stability test. The samples were centrifuged for 5 minutes at different speeds and visually examined in order to determine physical changes such as creaming (accumulation of lipid globules at the surface) or phase changes (complete separation of the oil and aqueous phases).

The results obtained are presented in Table 2 below.

TABLE 2

| | Macroscopic | Globule size | Centrifugation |
|---|---|---|---|
| Compositions | aspect | (μm) | performance |
| E1 | Very white and | 2.9 | + (no change) |
| | homogeneous | | |
| | emulsion | | |
| E2 | Very white and | 7.8 | + (no change) |
| | homogeneous | | |
| | emulsion | | |
| E3 | Thick, very | 3.4 | + (no change) |
| | white and | | |
| | homogeneous | | |
| | emulsion | | |
| E4 | Very white and | 2.9 | + (no change) |
| | homogeneous | | |
| | emulsion | | |
| E5 | Very white and | 3.3 | + (no change) |
| | homogeneous | | |
| | emulsion | | |
| EC1 | Immediate | Measurement | Measurement not |
| | phase change | not performed: | performed: in |
| | | in step a) of | step a) of the |
| | | the method, | method, the |
| | | the | intermediate |
| | | intermediate | composition |
| | | composition | immediately |
| | | immediately | undergoes a |
| | | undergoes a | phase change: an |
| | | phase change: | intermediate |
| | | an | emulsion cannot |
| | | intermediate | be produced |
| | | emulsion | |
| | | cannot be | |
| | | produced | |
| EC2 | Rapid phase | Measurement | Measurement not |
| | change | not performed: | performed: the |
| | | the emulsion | emulsion rapidly |
| | | rapidly | undergoes a |
| | | undergoes a | phase change |
| | | phase change | |
| EC3 | Yellowish | 33.0 | The emulsion |
| | emulsion | | undergoes a |
| | | | phase change |

In the manufacturing conditions according to the invention, examples E1 to E5 illustrate emulsions containing between 18.8% and 40% oil and a small amount of surfactant between 0.14% and 0.375%.

The measurement of the globule sizes reflects characteristic sizes generally accepted for conventional emulsions widely containing more surfactants (2 to 50 μm).

In addition, the evaluation of the stability in centrifugation shows the absence of any physical changes for examples E1 to E5 according to the invention, when they contain little surfactant.

Comparative example EC1, in which the proportion of aqueous phase over oil phase in the intermediate composition is outside of the ranges claimed (step a) of the method), shows that it is thus not possible to obtain an intermediate emulsion since an immediate phase change is observed.

Comparative example EC2, in which the proportions of oil phase and surfactant in the final carrier are outside of the ranges claimed, but for which an intermediate emulsion according to step a) of the method of the invention is used, shows that the final carrier obtained has a rapid phase change, indicating its instability.

Comparative example EC3, in which a fat-soluble surfactant has been incorporated in the oil phase in addition to the water-soluble surfactant (PVA) in the aqueous phase shows, by comparison with example E1, that the final emulsion EC3, while technically capable of being produced, undergoes a phase change in the centrifugation rupture test, indicating its instability. It also has a large globule size than in example E1.

1. Carrier in the form of an oil-in-water (O/W) emulsion comprising at least one aqueous phase and at least one oil phase, in which said aqueous phase comprises at least one water-soluble surfactant and at least one water-soluble viscosifying agent, characterized in that:
the oil phase represents at least 10% by weight of the total weight of the carrier,
the water-soluble surfactant represents 0.1% to 0.5% by weight of the total weight of the carrier.

2. Carrier according to claim 1, characterized in that the oil phase is free of surfactant.

3. Carrier according to claim 1, characterized in that the water-soluble surfactant represents 0.2% to 0.4% by weight of the total weight of the carrier.

4. Carrier according to claim 1, characterized in that the oil phase represents no more than 60% by weight of the total weight of the carrier.

5. Carrier according to claim 4, characterized in that the oil phase represents around 15% to 50% by weight of the total weight of the carrier.

6. Carrier according to claim 1, characterized in that the water-soluble surfactant is chosen from polysorbates, lecithins, polyethylene glycol derivatives, sorbitan esters, polyoxyethylene-polyoxypropylene copolymers and water-soluble polyvinyl alcohols.

7. Carrier according to claim 6, characterized in that the water-soluble surfactant is a water-soluble polyvinyl alcohol (PVA).

8. Carrier according to claim 7, characterized in that the water-soluble polyvinyl alcohol (PVA) is partially hydrolyzed.

9. Carrier according to claim 1, characterized in that the water-soluble viscosifying agent is chosen from gums, the glycosaminoglycans, cellulose and derivatives thereof, and acrylic polymers or carbomers.

10. Carrier according to claim 1, characterized in that the oil phase includes at least one oil chosen from the group consisting of mineral oils, vegetable oils, silicone oils, synthetic oils and fluorinated oils.

11. Carrier according to claim 10, characterized in that the oil is chosen from jojoba, avocado, sesame, sunflower, corn, soybean, safflower, grape seed, olive, almond, castor, moring a, coconut, palm, borage, rapeseed, wheat germ, linseed, primrose, argan calendula and cottonseed oils.

12. Carrier according to claim 1, characterized in that it includes one or more additives chosen from preservatives, antioxidants, isotonics, chelating agents, buffers, polymers, fillers, hydrophilic or lipophilic gelling agents, hydrophilic filters, hydrophilic compounds such as alcohols, odor absorbers, humectants and emollients.

13. Cosmetic and/or dermatological composition in the form of an oil-in-water emulsion, characterized in that it includes:
a carrier as defined according to claim 1, and
at least one active principle for cosmetic and/or dermatological use.

14. Composition according to claim 13, characterized in that the active principle is an active principle for dermatological use chosen from corticosteroids, antibiotics, antifungal agents, antiseptics, anti-parasitic agents, anti-herpetic agents, anti-acne agents, anti-pruritic agents, keratolitic agents, products for treating psoriasis, and products for treating atopic dermatitis.

15. Composition according to claim 13, characterized in that the active principle is a cosmetic and/or dermatological active principle chosen from proteins, amino acids, polyols, urea, sugars and sugar derivatives, water-soluble vitamins, plant-based extracts and hydroxy acids.

16. Method for preparing a vehicle as defined according to claim 1, including the following series of steps:
a) preparing an oil-in-water emulsion by introducing, in a reactor, at least one oil that will represent 10% to 60% by weight of the total weight of the final carrier, then adding an aqueous surfactant solution in an amount so that:
the mass ratio of the amount of aqueous phase in the first emulsion over the amount of oil phase in the first emulsion is between 1/3 and 1/1, and
the mass ratio of the amount of surfactant in the carrier (i.e. the final carrier obtained from step c) over the amount of oil in the carrier is between 0.007 and 0.020;
b) preparing an aqueous water-soluble viscosifying agent solution, of which the concentration is dependent on the viscosifying agent used and the intended use;
c) introduction of the first emulsion into the aqueous water-soluble viscosifying agent solution, in order to obtain a second emulsion constituting said carrier, in which the amount of first emulsion introduced into the aqueous water-soluble viscosifying agent solution is such that the oil phase in said carrier represents at least 10% by weight of the total weight of said carrier and the water-soluble surfactant represents 0.1% to 0.5% by weight of the total weight of said carrier.

17. Method according to claim 16, characterized in that the mass ratio of the amount of aqueous phase in the first emulsion over the amount of oil phase in the first emulsion is 1/2.

18. Carrier according to claim 2, characterized in that the water-soluble surfactant represents 0.2% to 0.4% by weight of the total weight of the carrier.
